(19) United States
(12) Patent Application Publication
(10) Pub. No.: US 2010/0057065 A1
(43) Pub. Date: Mar. 4, 2010

(54) METHOD FOR CRYOSPRAY ABLATION

(75) Inventor: WILLIAM S. KRIMSKY, Baltimore, MD (US)

Correspondence Address: Womble Carlyle Sandridge & Rice PLLC;Christopher E. Jeffers, Seventh Floor, 1401 Eye Street N.W., Washington, DC, 20005, US

(73) Assignee: Reset Medical, Inc., Baltimore, MD (US)

(21) Appl. No.: 12/554,148

(22) Filed: Sep. 4, 2009

Related U.S. Application Data
(60) Provisional application No. 61/094,164, filed on Sep. 4, 2008.

(51) Int. Cl. A61B 18/02

(52) U.S. Cl. 606/21

(57) ABSTRACT

Methods for treating oral, nasal, pharyngeal and/or laryngeal tissue in a subject are described. Methods of the invention may comprise spraying a tissue to be treated with a cryogen and/or using a cryogen to create an isotherm in proximity to the tissue to be treated.

### CROSS REFERENCE TO RELATED APPLICATIONS

This applications claims priority to U.S. provisional application for patent No. 61/094,164, filed Sep. 4, 2008, the entire contents of which are specifically incorporated herein in its entirety.

### FIELD OF THE INVENTION

The invention relates to methods of treating conditions of tissue in the head and neck, for example, oral, nasal, pharyngeal and/or laryngeal tissue.

### BACKGROUND

A variety of malignancies can occur in head and neck tissue. The most commonly observed malignancy is squamous cell carcinoma, which arises in the cells that line the inside of the nose, mouth and throat. Other less common types of head and neck cancers include salivary gland tumors, lymphomas and sarcomas.

Head and neck malignancies, for example, carcinomas, sarcomas and/or lymphomas, are a pressing problem in the healthcare field. The problem afflicts both adults and children. Most all adult nasopharyngeal malignancies are carcinomas, while only about 20-35% of nasopharyngeal malignancies are carcinomas in children and are mostly rhabdomyosarcomas or lymphomas.

In addition to cancer of the head and neck, a significant number of patients suffer from problems associated with either benign or dysplastic tissue in this area, such as lymphoid tissue. For example, adenoids and tonsils may be prone to chronic infection or cause chronic infection of the ear, especially in children. Inflammation of these tissue can block the airway, thus, leading to difficulty in breathing, excessive snoring, sleep apnea, and in extreme cases, chronic comorbid conditions.

Traditional approaches for removing lesions or cancer include surgical resection, radiotherapy, and chemotherapy. Adenoidectomy and tonsillectomy involves the tissue being curetted, cauterized, lasered, or otherwise ablated. All currently available treatment modalities are associated with complications and pain. The survival rates of patients with nasopharyngeal carcinoma range from 40-50% when treated with radiotherapy alone, or 55-80% when a combination of radiation therapy and chemotherapy is administered.

For instance, narrowing of the glottic and subglottic areas from either congenital or acquired stenosis remains difficult to manage despite a variety of surgical and endoluminal approaches. Surgical interventions, such as resection or tracheostomy, and endoluminal interventions, such as dilatation, stenting, and ablation, are often combined with one another with variable and inconsistent results. Given the potential complications and inconsistent outcomes of surgery, alternative approaches to managing glottic and subglottic stenoses are needed. Although prior work with cryoprobes in the aerodigestive tract demonstrated some normalization of the mucosa and a more controlled wound response, there remained issues with mechanical injury and the degree to which the temperature of the target tissue could be reduced. Non-contact spray cryotherapy is a novel modality that has been used extensively in the gastrointestinal (GI) tract. Studies in the GI tract have demonstrated eradication of intramucosal carcinoma effective treatment of HPV infection and hemostasis providing for a submucosal injury without the potential for mechanical injury and normalization of the mucosa with a controlled wound response. Additionally, cryotherapy has also been used as adjuvant therapy in patients undergoing transoral resection of early glottic cancers and has been shown to improve voice quality in these patients.

Contact cryotherapy using a cooled cryoprobe has been used to treat conditions of head and neck tissue for example, severe nasal vasomotor instability (Strome, Ear Nose Throat J. 1990 December; 69(12):839-42) and as an adjunct to other treatments, for example, to laser resection in glottic cancer (Knott et al., Arch Otolaryngol Head Neck Surg. 2006 November; 132(11):1226-30). Contact cryotherapy is associated with several disadvantages including the tendency of the cryoprobe to stick to the treated tissue and cause unwanted tissue injury.

As the currently available treatment modalities are all associated with various drawbacks, there remains a need in the art for improved materials and methods for treating tissue of the head and neck. This need and others are met by the present invention.

### SUMMARY OF THE INVENTION

The present invention provides materials and methods for treatment of oral, nasal, pharyngeal and/or laryngeal tissue. In some embodiments, methods of the invention may comprise spraying the tissue with one or more cryogens, and/or using one or more cryogens to create an isotherm in proximity to the tissue. Any suitable cryogen may be used. Preferably a cryogen will be a liquid having a boiling point temperature lower than the freezing point the oral, nasal, pharyngeal and/or laryngeal tissue to be treated. In some embodiments, a cryogen may be a liquefied gas, for example, oxygen, a nitrogen oxide, nitrogen and argon. In some embodiments, the cryogen may be carbon dioxide. The temperature of the isotherm can be adjusted by controlling the rate at which cryogen is delivered. The temperature of the isotherm may be sufficiently depressed from normal body temperature to generate the desired response. Suitable examples of temperatures may include, but are not limited to, from about 4° C. to about the boiling point of the cryogen. Typically, the oral, nasal, pharyngeal and/or laryngeal tissue may be sprayed with cryogen or is in proximity to the isotherm for a period of time sufficient to initiate a tissue response. In some embodiments, oral, nasal, pharyngeal and/or laryngeal tissue may be sprayed with cryogen or may be maintained in proximity to the isotherm for a period of time sufficient to freeze the tissue. In one embodiment, a proximal end of a catheter is connected to a cryogen source and a distal end of the catheter is placed in proximity to the oral, nasal, pharyngeal and/or laryngeal tissue and cryogen flows from the source through the distal end to the tissue. Optionally, the distal end of the catheter is guided to the oral, nasal, pharyngeal and/or laryngeal tissue using a guiding device. Any suitable guiding device may be used. One example of a guiding device might comprise a video camera and the distal end of the guiding device and/or the catheter may be guided to the oral, nasal, pharyngeal and/or laryngeal tissue by observing the distal end of the catheter and/or guiding device on a video monitor. Any type of tissue may be treated using the materials and methods of the invention. In some embodiments, oral, nasal, pharyngeal and/or laryngeal tissue to be treated may comprise unwanted tissue, for example, tissue comprising cancerous cells.

In one embodiment, the present invention provides materials and methods for treating a lesion in oral, nasal, pharyngeal and/or laryngeal tissue. Such methods typically comprise spraying oral, nasal, pharyngeal and/or laryngeal tissue comprising a lesion with a cryogen or using the cryogen to create an isotherm in proximity to the tissue comprising a lesion. Any suitable cryogen may be used. Preferably a cryogen will be a liquid having a boiling point temperature lower than the freezing point the oral, nasal, pharyngeal and/or laryngeal tissue comprising a lesion. In some embodiments, a cryogen may be a liquefied gas, for example, oxygen, a nitrogen oxide, nitrogen and argon. In some embodiments, the cryogen may be carbon dioxide. The temperature of the isotherm can be adjusted by controlling the rate at which cryogen is delivered. The temperature of the isotherm may be sufficiently depressed from normal body temperature to generate the desired response. Suitable examples of temperatures may include, but are not limited to, from about 4° C. to about the boiling point of the cryogen. Typically, the oral, nasal, pharyngeal and/or laryngeal tissue comprising a lesion may be sprayed with cryogen or is in proximity to the isotherm for a period of time sufficient to initiate a tissue response. In some embodiments, oral, nasal, pharyngeal and/or laryngeal tissue comprising a lesion may be sprayed with cryogen or may be maintained in proximity to the isotherm for a period of time sufficient to freeze the tissue. In one embodiment, a proximal end of a catheter is connected to a cryogen source and a distal end of the catheter is placed in proximity to the oral, nasal, pharyngeal and/or laryngeal tissue comprising a lesion and cryogen flows from the source through the distal end to the tissue comprising a lesion. Optionally, the distal end of the catheter is guided to the oral, nasal, pharyngeal and/or laryngeal tissue comprising a lesion using a guiding device. Any suitable guiding device may be used. One example of a guiding device might comprise a video camera and the distal end of the guiding device and/or the catheter may be guided to the oral, nasal, pharyngeal and/or laryngeal tissue comprising a lesion by observing the distal end of the catheter and/or guiding device on a video monitor. Any type of tissue comprising a lesion may be treated using the materials and methods of the invention. In some embodiments, oral, nasal, pharyngeal and/or laryngeal tissue comprising a lesion may comprise cancerous cells.

In one embodiment, the present invention provides materials and methods for stimulating a response in oral, nasal, pharyngeal and/or laryngeal tissue. In some embodiments, methods of the invention may comprise spraying the tissue with one or more cryogens, and/or using one or more cryogens to create an isotherm in proximity to the tissue. Any suitable cryogen may be used. Preferably a cryogen will be a liquid having a boiling point temperature lower than the freezing point the oral, nasal, pharyngeal and/or laryngeal tissue in which a response is to be stimulated. In some embodiments, a cryogen may be a liquefied gas, for example, oxygen, a nitrogen oxide, nitrogen and argon. In some embodiments, the cryogen may be carbon dioxide. The temperature of the isotherm can be adjusted by controlling the rate at which cryogen is delivered. The temperature of the isotherm may be sufficiently depressed from normal body temperature to generate the desired response. Suitable examples of temperatures may include, but are not limited to, from about 4° C. to about the boiling point of the cryogen. Typically, the oral, nasal, pharyngeal and/or laryngeal tissue in which a response is to be stimulated may be sprayed with cryogen or is in proximity to the isotherm for a period of time sufficient to initiate a tissue response. In some embodiments, oral, nasal, pharyngeal and/or laryngeal tissue in which a response is to be stimulated may be sprayed with cryogen or may be maintained in proximity to the isotherm for a period of time sufficient to freeze the tissue. In one embodiment, a proximal end of a catheter is connected to a cryogen source and a distal end of the catheter is placed in proximity to the oral, nasal, pharyngeal and/or laryngeal tissue in which a response is to be stimulated and cryogen flows from the source through the distal end to the tissue. Optionally, the distal end of the catheter is guided to the oral, nasal, pharyngeal and/or laryngeal tissue in which a response is to be stimulated using a guiding device. Any suitable guiding device may be used. One example of a guiding device might comprise a video camera and the distal end of the guiding device and/or the catheter may be guided to the oral, nasal, pharyngeal and/or laryngeal tissue in which a response is to be stimulated by observing the distal end of the catheter and/or guiding device on a video monitor. A response may be stimulated in any type of oral, nasal, pharyngeal and/or laryngeal tissue using the materials and methods of the invention. In some embodiments, oral, nasal, pharyngeal and/or laryngeal tissue in which a response is to be stimulated may comprise unwanted tissue, for example, tissue comprising cancerous cells.

In some embodiments, the present invention provides materials and methods for treating an infection in oral, nasal, pharyngeal and/or laryngeal tissue. Such methods may comprise spraying an infected oral, nasal, pharyngeal and/or laryngeal tissue with a cryogen or using the cryogen to create an isotherm in proximity to the tissue. Any suitable cryogen may be used. Preferably a cryogen will be a liquid having a boiling point temperature lower than the freezing point the infected oral, nasal, pharyngeal and/or laryngeal tissue. In some embodiments, a cryogen may be a liquefied gas, for example, oxygen, a nitrogen oxide, nitrogen and argon. In some embodiments, the cryogen may be carbon dioxide. The temperature of the isotherm can be adjusted by controlling the rate at which cryogen is delivered. The temperature of the isotherm may be sufficiently depressed from normal body temperature to generate the desired response. Suitable examples of temperatures may include, but are not limited to, from about 4° C. to about the boiling point of the cryogen. Typically, the infected oral, nasal, pharyngeal and/or laryngeal tissue may be sprayed with cryogen or is in proximity to the isotherm for a period of time sufficient to initiate a tissue response. In some embodiments, infected oral, nasal, pharyngeal and/or laryngeal tissue may be sprayed with cryogen or may be maintained in proximity to the isotherm for a period of time sufficient to freeze all or a portion of the tissue. In one embodiment, a proximal end of a catheter is connected to a cryogen source and a distal end of the catheter is placed in proximity to the infected oral, nasal, pharyngeal and/or laryngeal tissue and cryogen flows from the source through the distal end to the tissue. Optionally, the distal end of the catheter is guided to the infected oral, nasal, pharyngeal and/or laryngeal tissue using a guiding device. Any suitable guiding device may be used. One example of a guiding device might comprise a video camera and the distal end of the guiding device and/or the catheter may be guided to the infected oral, nasal, pharyngeal and/or laryngeal tissue by observing the distal end of the catheter and/or guiding device on a video monitor. An infection may be treated in any type of oral, nasal, pharyngeal and/or laryngeal tissue using the materials and methods of the invention.

In some embodiments, materials and methods of the invention may be used to treat unwanted and/or unnecessary oral, nasal, pharyngeal and/or laryngeal tissue. Such methods may comprise spraying an unwanted oral, nasal, pharyngeal and/or laryngeal tissue with a cryogen or using the cryogen to create an isotherm in proximity to the tissue. Any suitable cryogen may be used. Preferably a cryogen will be a liquid having a boiling point temperature lower than the freezing point the unwanted oral, nasal, pharyngeal and/or laryngeal tissue. In some embodiments, a cryogen may be a liquefied gas, for example, oxygen, a nitrogen oxide, nitrogen and argon. In some embodiments, the cryogen may be carbon dioxide. The temperature of the isotherm can be adjusted by controlling the rate at which cryogen is delivered. The temperature of the isotherm may be sufficiently depressed from normal body temperature to generate the desired response. Suitable examples of temperatures may include, but are not limited to, from about 4° C. to about the boiling point of the cryogen. Typically, the unwanted oral, nasal, pharyngeal and/or laryngeal tissue may be sprayed with cryogen or is in proximity to the isotherm for a period of time sufficient to initiate a tissue response. In some embodiments, unwanted oral, nasal, pharyngeal and/or laryngeal tissue may be sprayed with cryogen or may be maintained in proximity to the isotherm for a period of time sufficient to freeze all or a portion of the tissue. In one embodiment, a proximal end of a catheter is connected to a cryogen source and a distal end of the catheter is placed in proximity to the unwanted oral, nasal, pharyngeal and/or laryngeal tissue and cryogen flows from the source through the distal end to the tissue. Optionally, the distal end of the catheter is guided to the unwanted oral, nasal, pharyngeal and/or laryngeal tissue using a guiding device. Any suitable guiding device may be used. One example of a guiding device might comprise a video camera and the distal end of the guiding device and/or the catheter may be guided to the unwanted oral, nasal, pharyngeal and/or laryngeal tissue by observing the distal end of the catheter and/or guiding device on a video monitor. Unwanted and/or unnecessary tissue may be treated in any type of oral, nasal, pharyngeal and/or laryngeal tissue using the materials and methods of the invention. Any type of unwanted and/or unnecessary tissue may be treated using materials and methods of the invention, for example, unwanted and/or unnecessary tissue may comprise a lesion, and/or may comprise cancerous cells. In one embodiment, unwanted tissue may comprise tissue at the margin of a surgical site. Thus, the present invention also provides materials and methods for clearing and/or enhancing surgical margins.

In some embodiments, materials and methods of the invention may be used to modulate an immune response in oral, nasal, pharyngeal and/or laryngeal tissue. Such methods may comprise spraying oral, nasal, pharyngeal and/or laryngeal tissue with a cryogen or using the cryogen to create an isotherm in proximity to the tissue in which the immune response is to be modulated. Any suitable cryogen may be used. Preferably a cryogen will be a liquid having a boiling point temperature lower than the freezing point the oral, nasal, pharyngeal and/or laryngeal tissue in which the immune response is to be modulated. In some embodiments, a cryogen may be a liquefied gas, for example, oxygen, a nitrogen oxide, nitrogen and argon. In some embodiments, the cryogen may be carbon dioxide. The temperature of the isotherm can be adjusted by controlling the rate at which cryogen is delivered. The temperature of the isotherm may be sufficiently depressed from normal body temperature to generate the desired response. Suitable examples of temperatures may include, but are not limited to, from about 4° C. to about the boiling point of the cryogen. Typically, the oral, nasal, pharyngeal and/or laryngeal tissue in which the immune response is to be modulated may be sprayed with cryogen or is in proximity to the isotherm for a period of time sufficient to initiate a tissue response. In some embodiments, oral, nasal, pharyngeal and/or laryngeal tissue may be sprayed with cryogen or may be maintained in proximity to the isotherm for a period of time sufficient to freeze all or a portion of the tissue in which the immune response is to be modulated. In one embodiment, a proximal end of a catheter is connected to a cryogen source and a distal end of the catheter is placed in proximity to the oral, nasal, pharyngeal and/or laryngeal tissue and cryogen flows from the source through the distal end to the tissue in which the immune response is to be modulated. Optionally, the distal end of the catheter is guided to the oral, nasal, pharyngeal and/or laryngeal tissue in which the immune response is to be modulated using a guiding device. Any suitable guiding device may be used. One example of a guiding device might comprise a video camera and the distal end of the guiding device and/or the catheter may be guided to the oral, nasal, pharyngeal and/or laryngeal tissue in which the immune response is to be modulated by observing the distal end of the catheter and/or guiding device on a video monitor. An immune response can be modulated in any type of oral, nasal, pharyngeal and/or laryngeal tissue using the materials and methods of the invention, for example, oral, nasal, pharyngeal and/or laryngeal tissue may comprise unwanted tissue, one or more lesions, and/or cancerous cells.

In some embodiments, materials and methods of the invention may be used to treat benign or malignant tumors or lesions, dysplastic tissue, and/or neoplastic tissue or disease in a subject in need thereof. Such methods may include spraying oral, nasal, pharyngeal and/or laryngeal tissue in the subject with a cryogen, or using the cryogen to create an isotherm in proximity to oral, nasal, pharyngeal and/or laryngeal tissue, wherein the oral, nasal, pharyngeal and/or laryngeal tissue comprises the benign or malignant tumor or lesion and/or neoplastic disease tissue. Any suitable cryogen may be used. Preferably a cryogen will be a liquid having a boiling point temperature lower than the freezing point the oral, nasal, pharyngeal and/or laryngeal tissue comprising a benign or malignant tumor or lesion and/or neoplastic disease. In some embodiments, a cryogen may be a liquefied gas, for example, oxygen, a nitrogen oxide, nitrogen and argon. In some embodiments, the cryogen may be carbon dioxide. The temperature of the isotherm can be adjusted by controlling the rate at which cryogen is delivered. The temperature of the isotherm may be sufficiently depressed from normal body temperature to generate the desired response. Suitable examples of temperatures may include, but are not limited to, from about 4° C. to about the boiling point of the cryogen. Typically, the oral, nasal, pharyngeal and/or laryngeal tissue comprising benign or malignant tumors or lesions, dysplastic tissue, and/or neoplastic tissue or disease may be sprayed with cryogen or is in proximity to the isotherm for a period of time sufficient to initiate a tissue response. In some embodiments, oral, nasal, pharyngeal and/or laryngeal tissue may be sprayed with cryogen or may be maintained in proximity to the isotherm for a period of time sufficient to freeze all or a portion of the tissue comprising benign or malignant tumors or lesions, dysplastic tissue, and/or neoplastic tissue or disease. In one embodiment, a proximal end of a catheter is connected to a cryogen source and a distal end of the catheter is placed in proximity to the oral, nasal, pharyngeal and/or laryngeal tissue comprising benign or malignant tumors or lesions, dysplastic tissue, and/or neoplastic tissue or disease and cryogen flows from the source through the distal end to the tissue comprising benign or malignant tumors or lesions, dysplastic tissue, and/or neoplastic tissue or disease. Optionally, the distal end of the catheter is guided to the oral, nasal, pharyngeal and/or laryngeal tissue comprising benign or malignant tumors or lesions, dysplastic tissue, and/or neoplastic tissue or disease using a guiding device. Any suitable guiding device may be used. One example of a guiding device might comprise a video camera and the distal end of the guiding device and/or the catheter may be guided to the oral, nasal, pharyngeal and/or laryngeal tissue comprising benign or malignant tumors or lesions, dysplastic tissue, and/or neoplastic tissue or disease by observing the distal end of the catheter and/or guiding device on a video monitor. Any oral, nasal, pharyngeal and/or laryngeal tissue comprising benign or malignant tumors or lesions, dysplastic tissue, and/or neoplastic tissue or disease can be treated using the materials and methods of the invention. In some embodiments, benign or malignant tumors or lesions, dysplastic tissue, and/or neoplastic tissue may be left after a surgical procedure removing other benign or malignant tumors or lesions, dysplastic tissue, and/or neoplastic tissue. Thus, methods of the invention may be used in conjunction with other surgical procedures. For example, benign or malignant tumors or lesions, dysplastic tissue, and/or neoplastic tissue may be removed using standard surgery and the margins of the surgical site may be cleared and/or enhanced by further treatment with cryospray.

In some embodiments, the present invention provides methods of stimulating cartilage and/or bone growth in oral, nasal, pharyngeal and/or laryngeal tissue. Such methods typically entail injuring the cartilage and/or bone with a cryogen, for example, with a liquefied gas such as liquid nitrogen under conditions resulting in stimulation of chondrogenesis. Cartilage and/or bone may be injured by spraying oral, nasal, pharyngeal and/or laryngeal tissue comprising cartilage and or bone with a cryogen, or using the cryogen to create an isotherm in proximity to oral, nasal, pharyngeal and/or laryngeal tissue comprising cartilage and/or bone. Any suitable cryogen may be used. Preferably a cryogen will be a liquid having a boiling point temperature lower than the freezing point the oral, nasal, pharyngeal and/or laryngeal tissue comprising cartilage and/or bone. In some embodiments, a cryogen may be a liquefied gas, for example, oxygen, a nitrogen oxide, nitrogen and argon. In some embodiments, the cryogen may be carbon dioxide. The temperature of the isotherm can be adjusted by controlling the rate at which cryogen is delivered. The temperature of the isotherm may be sufficiently depressed from normal body temperature to generate the desired response. Suitable examples of temperatures may include, but are not limited to, from about 4° C. to about the boiling point of the cryogen. Typically, the oral, nasal, pharyngeal and/or laryngeal tissue comprising cartilage and/or bone may be sprayed with cryogen or is in proximity to the isotherm for a period of time sufficient to initiate a tissue response. In some embodiments, oral, nasal, pharyngeal and/or laryngeal tissue may be sprayed with cryogen or may be maintained in proximity to the isotherm for a period of time sufficient to freeze all or a portion of the tissue comprising cartilage and/or bone. In one embodiment, a proximal end of a catheter is connected to a cryogen source and a distal end of the catheter is placed in proximity to the oral, nasal, pharyngeal and/or laryngeal tissue comprising cartilage and/or bone and cryogen flows from the source through the distal end to the tissue comprising cartilage and/or bone. Optionally, the distal end of the catheter is guided to the oral, nasal, pharyngeal and/or laryngeal tissue comprising cartilage and/or bone using a guiding device. Any suitable guiding device may be used. One example of a guiding device might comprise a video camera and the distal end of the guiding device and/or the catheter may be guided to the oral, nasal, pharyngeal and/or laryngeal tissue comprising cartilage and/or bone by observing the distal end of the catheter and/or guiding device on a video monitor. Any oral, nasal, pharyngeal and/or laryngeal tissue comprising cartilage and/or bone can be treated using the materials and methods of the invention.

In some methods of stimulating cartilage and/or bone growth in oral, nasal, pharyngeal and/or laryngeal tissue, cartilage and/or bone is sprayed with cryogen for a period of time sufficient to initiate a response in and/or freeze the cartilage and/or bone. Alternatively, the cartilage and/or bone may be in proximity to an isotherm having a temperature below the freezing point of the cartilage and/or bone for a period of time sufficient to initiate a response in and/or freeze the cartilage and/or bone. In some embodiments, the temperature of the cartilage and/or bone is reduced but the cartilage and/or bone is not frozen. This can be accomplished by creating an isotherm in proximity to the cartilage and/or bone to be treated, wherein the temperature of the isotherm is below that of the cartilage and/or bone and maintaining the cartilage and/or bone in proximity to the isotherm for a period of time sufficient to reduce the temperature of the cartilage and/or bone. In some embodiments, cartilage and/or bone is sprayed with cryogen for a period of time sufficient to damage a portion of the cartilage and/or bone. In some embodiments, a plurality of isotherms may be created in proximity to the cartilage and/or bone to stimulate chondrogenesis. For example, a first isotherm may be created at a first temperature and the cartilage and/or bone maintained in proximity to the first isotherm. The first isotherm may be removed and a second isotherm which may be at the same or different temperature may created and the cartilage and/or bone maintained in proximity to the second isotherm. A period of time may elapse between removal of the first isotherm and creation of the second isotherm. Any number of isotherms may be created and their temperatures may be the same or different. A period of time may elapse between the removal of one isotherm and the creation of a second or a second may be created by modifying (for example, by increasing or decreasing the temperature) a first with no period of time between. The temperature of the isotherm may be sufficiently depressed from normal body temperature to generate the desired response. Suitable examples of temperatures may include, but are not limited to, from about 4° C. to about the boiling point of the cryogen.

### BRIEF DESCRIPTION OF THE DRAWINGS

For the purpose of illustrating the invention, there is shown in the drawings a form which is presently preferred; it being understood, that this invention is not limited to the precise arrangements and instrumentalities shown.

FIG. 1A depicts an ENT scope or endoscope or other guiding device inserted into the nasopharyngeal cavity of a patient.

FIG. 1B is an enlarged view of FIG. 1A and shows the distal end of the ENT scope or endoscope or other guiding device and the catheter within the nasopharyngeal cavity.

FIG. 2A depicts a close-up of the distal end of an endoscope or other guiding device, light source, camera, extra lumen, and lumen with an inserted catheter having an opening for releasing cryogen spray.

FIG. 2B depicts a close-up of the distal end of an endoscope or other guiding device, light source, camera, extra lumen, and lumen with an inserted catheter having a lateral opening for emitting directional cryogen spray.

FIG. 2C depicts a close-up of the distal end of an endoscope or other guiding device, light source, camera, extra lumen, and lumen with an inserted catheter having a laterally-disposed, cone-shaped structure for directing cryogen spray.

FIG. 3 is a schematic view of an apparatus for use in nasopharyngeal cryosurgery.

FIG. 4A is a photograph taken with an endoscope camera showing tracheal stenosis in a 43 year old female before treatment (pre-treatment with tracheal lumen is 5-6 mm). The circled area shows the lumen of the trachea before treatment.

FIG. 4B is a photograph taken with an endoscope camera showing the results of cryotherapy treatment of tracheal stenosis in a 43 year old female 10 days post-treatment.

FIG. 4C is a photograph taken with an endoscope camera showing the results of cryotherapy treatment of tracheal stenosis in a 43 year old female 4 weeks post-treatment demonstrating restoration of complete tracheal lumen patency.

FIG. 5A is a photograph taken with an endoscope camera showing glottic stricture and vocal cord stenosis in a 74 year old female before treatment (pre-treatment with glottic opening of 4-5 mm).

FIG. 5B is a photograph taken with an endoscope camera during cryotherapy treatment of glottic stricture and vocal cord stenosis in a 74 year old female.

FIG. 5C is a photograph taken with an endoscope camera of the results of cryotherapy on glottic stricture and vocal cord stenosis in a 74 year old female 6 weeks post-treatment demonstrating complete lumen patency.

FIG. 6A is a photograph taken with an endoscope camera showing tracheobronchiomalacia in a 33 year old female before treatment.

FIG. 6B is a photograph taken with an endoscope camera of the treated tracheobronchiomalacia in a 33 year old female 6 months post-treatment.

### DETAILED DESCRIPTION

The present invention provides materials and methods for treating oral, nasal, pharyngeal and/or laryngeal tissue. In some embodiments, the invention relates to a method of treating or preventing abnormal or pathogenic conditions in oral, nasal, pharyngeal and/or laryngeal tissue. As used herein, the phrase “oral, nasal, pharyngeal and/or laryngeal tissue” includes tissues of the mouth, nose, nasal cavity, sinuses, nasopharynx, oropharynx, pharynx, and larynx including the supraglottis, glottis and subglottis. Such tissues can include muscle, blood vessels, lymphatic tissue, epithelial, mucosal and submucosal tissue, cartilage, bone, nerve, and other connective tissue. Target tissues may be abnormal, diseased, damaged, or unwanted tissue. As used herein, the terms “target area”, “target tissue” and “tissue to be treated” refer to that portion of healthy, diseased, damaged or unwanted tissue to which a cryogen is, is to be, or has been applied.

The methods of the invention may be particularly useful for conditions relating to the controlled injury and/or ablation of oral, nasal, pharyngeal and/or laryngeal tissue including but not limited to cancers (e.g., cancer of the larynx, mouth, nose, sinuses, salivary glands, throat and lymph nodes in throat), nodules (e.g., nodules of the vocal fold) lesions (e.g., viral lesions) and infected tissue.

Methods of the invention can be carried out with a catheter alone or in combination with a guiding device, such as an endoscope (e.g., a naso-laryngoscope). A camera or other viewing device can also be used if visualization of the target tissue is desired and the tissue is not otherwise easily viewable. When an endoscope is used, it is also possible to deliver cryogen to the target tissue directly through an endoscope channel without a catheter. Any endoscope suitable for ear nose and throat (ENT) applications may be used in the methods of the invention. Such endoscopes are commercially available from, for example, Olympus Surgical & Industrial America Inc, Orangeburg, New York (e.g., models ENF-VT, ENF-V2, ENF-T3, ENF-P4, ENF-XP, ENF-GP, and ENF-L3 and imaging system EXERA II).

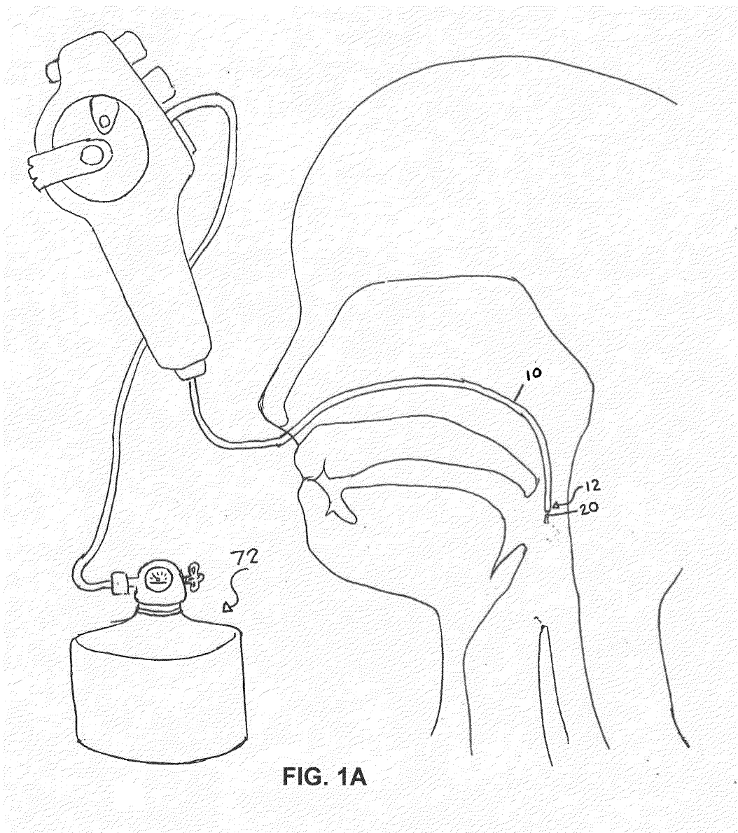

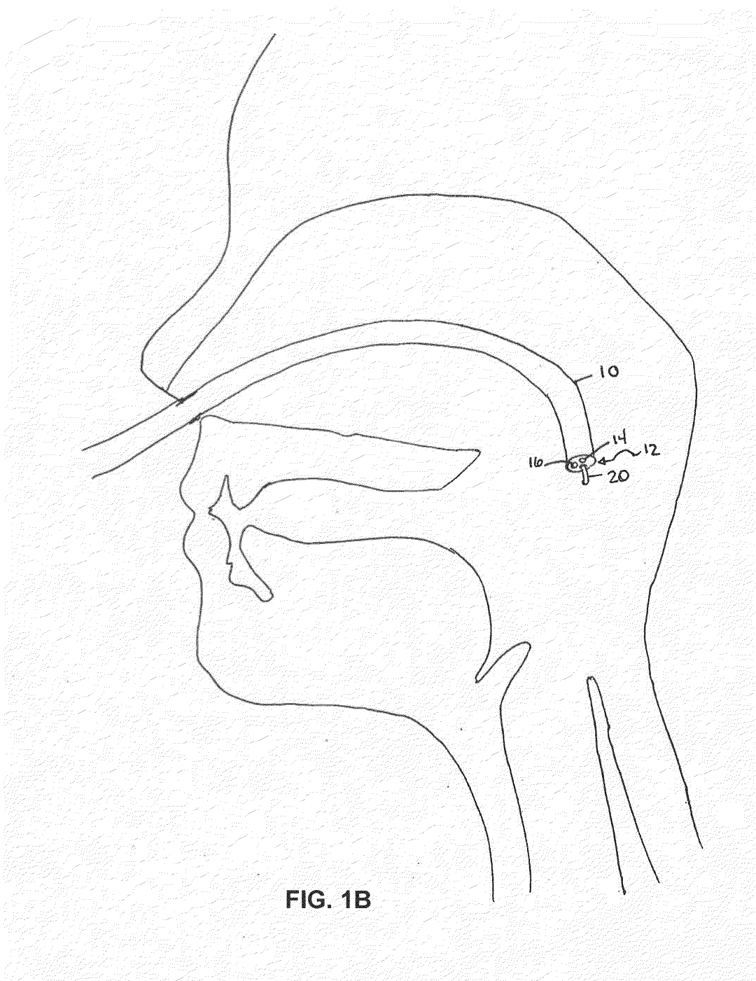

An apparatus for use in at least one method of the invention is shown in FIGS. 1A and 1B. The method can include directing cryogen to oral, nasal, pharyngeal and/or laryngeal tissue to be treated utilizing a guiding device, for example, an ENT scope or other suitable therapeutic endoscopic device, having a catheter inserted therethrough. Typically, a proximal end of a catheter is connected to a cryogen source and a distal end of the catheter is placed in proximity to the oral, nasal, pharyngeal and/or laryngeal tissue to be treated and cryogen flows from the source through the distal end to the tissue to be treated. Optionally, the distal end of the catheter is guided to the oral, nasal, pharyngeal and/or laryngeal tissue to be treated using a guiding device, e.g., an ENT scope. The catheter can be positioned to allow a cryogen fluid spray to be disposed adjacent a tissue to be treated (the “target tissue” or “target area”). The target oral, nasal, pharyngeal and/or laryngeal tissue can then be sprayed with a cryogen fluid spray or cryogen can be sprayed so as to create an isotherm in proximity to the tissue to be treated.

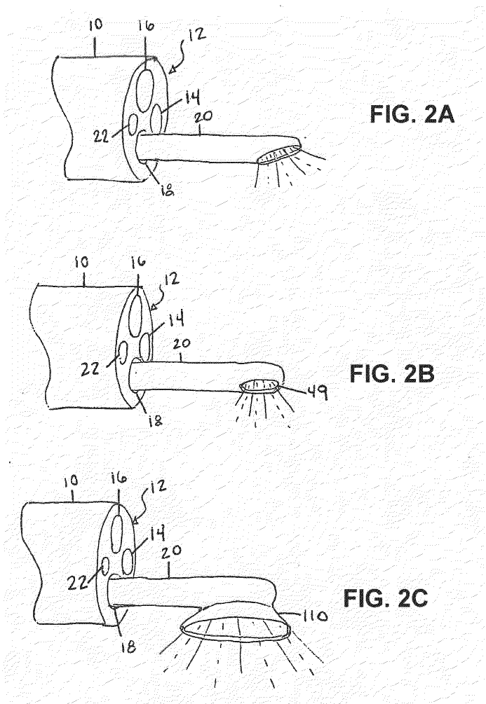

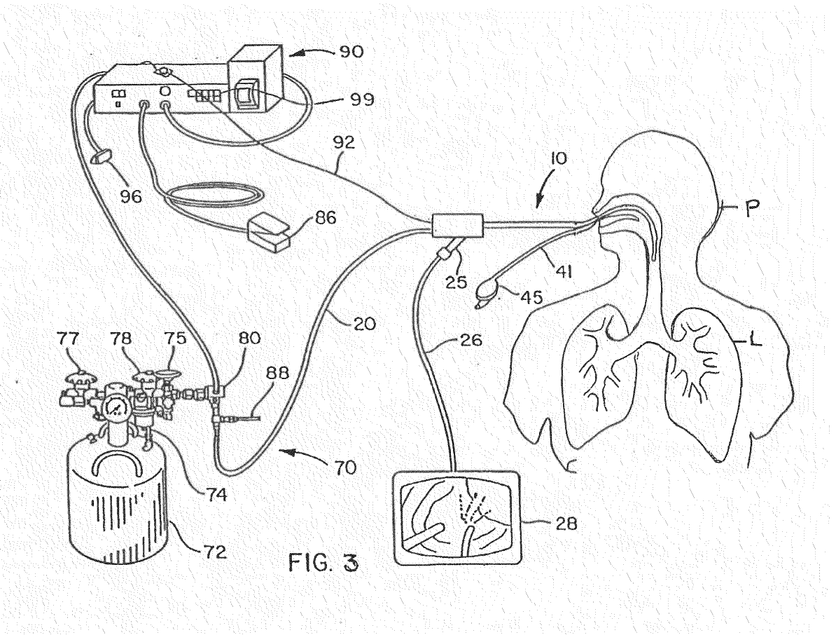

The methods of the present invention can be performed using a conventional naso-laryngoscope scope 10, as is illustrated in FIGS. 1A and 1B. The distal end 12 of such a naso-laryngoscope 10 is shown in FIGS. 2A, 2B, and 2C, showing an imaging camera lens 14, illuminating light 16, biopsy channel (bore or lumen) 18 with the catheter 20 therein. A naso-laryngoscope can contain an additional lumen 22. The image picked up at the lens 14 can be transferred via fiber optics to a monitoring camera 25 (FIG. 3) which sends TV signals via a cable 26 to a monitor 28, where the procedure can be directly visualized by a physician or other observer. The physician or operator can perform the methods of the invention on oral, nasal, pharyngeal and/or laryngeal tissue with the aid of this visualization.

Alternatively, if the tissue to be treated is readily visible and accessible, the catheter may be used without a scope. In lieu of an ENT scope, a simpler guiding device (e.g., a flexible tube or sleeve) for directing the distal end of the catheter towards the lesion may be used. Preferably, the guiding device helps to avoid direct contact between the tissue and catheter. In some embodiments, a guiding device may not be required. In such cases, the operator may direct the cryogen flow by directly controlling the catheter. In such instances, the operator may employ an insulating material, for example, a cloth such as a gauze pad, insulated gloves and the like, to grasp the catheter and direct the cryogen flow. Other commonly used surgical tools, such as forceps and/or hemostats may also be used to grasp the catheter to direct the cryogen flow.

A catheter 20 can be disposed through lumen 18 of an ENT scope or other guiding device. The size of the catheter should be selected to fit within the guiding device, for example, the narrow diameter of the working channel of an ENT scope. However, larger or smaller catheters can be used. For example, a PENTAX naso-pharyngo-laryngoscope VNL-1530T has a 2.0 mm working channel and a 300 mm working length. Any appropriate catheter sized to fit within the working channel (i.e., with a diameter of less than 2.0 mm) may be used if a VNL-1530T is employed.

The catheter 20 may protrude from the distal end 12 of the ENT scope or other guiding device 10 and extends through the scope or other guiding device to the proximal end 30 where a physician's hand can guide the catheter 20. As used herein, the terms “proximal” and “distal” respectively refer to locations closer to and farther away from the cryogen source along the length of the fluid connections and catheter extending therefrom. By way of example, the proximal end of a catheter or endoscope or other guiding device will generally remain outside of a patient during use, while the distal end of said catheter or endoscope or other guiding device will be inserted into the patient. As seen in the monitor image 28 of FIG. 3, the distal end 12 of the catheter 20 may be bent at an angle.

The catheter 20 can be coupled to a cryogen source, such as a container 72 filled with liquid nitrogen or other liquefied gas LG. As used in the present specification, “gas” in the phrase “liquefied gas” means any fluid which is physiologically acceptable and which has a sufficiently low boiling point to allow the cryotherapy of the present invention. For example, such boiling point is preferably below about −150° C. Examples of such gases include nitrogen, as it is readily available, and argon. Such gases may also be referred to as a “cryogen.” However, the term “cryogen” also refers to any fluid whether in liquid or gas form that is or was sufficiently cold to allow the cryotherapy.

FIG. 3 shows schematically the catheter 20 connected to a source of cryogen and inserted into the working channel of naso-laryngoscope 10. The pressure within the container can be controlled by the physician or other provider to permit adequate spray from the distal end of the catheter. Any suitable means may be used, for example, a pressure-building loop as described in U.S. Pat. No. 7,025,762 to Johnston. Additional embodiments and further details regarding the apparatus are also described in U.S. Pat. No. 7,025,762, which is hereby incorporated by reference in its entirety.

With reference to FIG. 3, a system for use in the methods of the invention 70 is described. In this system, a pressurized gas tank 72 is employed. Tank 72 may be equipped with a pressure building coil or tube 74 for maintaining pressure. In the tube arrangement shown, the valve 75 is hand operated, however, the valve could be automatic and would start circulating liquid through the tube or a coil once the pressure drops to unacceptable levels in the tank and to stop circulating once the pressure returns to normal. The tank 72 is equipped with a head gas valve 77 for relieving head pressure and a liquid nitrogen valve 78 which is opened to allow liquid nitrogen to flow to the solenoid valve 80 and then to catheter 20. System 70 may have a foot-pedal operated solenoid valve switch 86 which may actuate solenoid 80 between the tank 72 and catheter 20. The system may comprise an auxiliary bleeder vent or bleeder 88 positioned between the liquid nitrogen gas supply tank 72 and the catheter 20. The system may be equipped with electronic monitoring and recording components 90. The electronic components 90 may comprise a temperature sensor or probe 92 and/or timer 96 and/or and LED display 99 which may be used to display parameters (e.g., time, temperature etc) of the treatment.

The flow of liquefied gas from the cryogen source may be controlled using any structure known in the art, for example, a simple thumb-valve, a mechanical valve or an electromechanical valve. The valve may be controlled by a trigger mechanism, or the like, as could be readily envisioned and constructed by those of ordinary skill in the art. In an embodiment, an electrically operated solenoid valve may be employed to deliver the liquefied gas to the catheter. The solenoid can be specifically adapted to function properly at low temperatures.

As the liquefied gas moves through the catheter 20, it starts to boil and cool gas rushes ahead to emerge from the distal end or catheter tip. The boiling point of nitrogen is about −196° C. Thus, when nitrogen is used as the cryogen, low pressure liquid moving through the catheter will be less than −150° C. The amount of boiling in the catheter 20 depends on the mass and thermal capacity of the catheter. Since the catheter is of small diameter and mass, the amount of boiling can be small. After the catheter is cooled to a low temperature, and becomes filled with liquefied gas, the liquefied gas reaches the distal end of the catheter 20 near the distal end of endoscope 12 and begins to spray out of the catheter onto the appropriate target tissue.

In some methods, liquid cryogen is not sprayed directly upon a target tissue. Instead, the cryogen is delivered through the distal end of the catheter at a rate such that the cryogen undergoes liquid to gas phase transition before coming into contact with the target tissue. In effect, cryogen is delivered to a site of treatment as a cold gas. The cold gas causes a reduction in the ambient temperature of the region around the distal end of the catheter. As used herein, “isotherm” indicates a region of reduced ambient temperature. Thus, delivery of cryogen can be used to reduce the ambient temperature at a site to be treated. The temperature of the isotherm can be maintained at any desired value by increasing (to reduce temperature) or decreasing (to increase temperature) the rate at which cryogen is delivered through the catheter and exits the distal end of the catheter. The catheter and/or the guiding device may be equipped with a temperature sensor in order to monitor the temperature of an isotherm. Optionally, the data from the temperature sensor can be displayed on the control panel. In some embodiments, the data from the temperature sensor is used to control a valve (for example, a solenoid valve as discussed above) that controls the rate of flow of cryogen through the catheter. In such embodiments, the desired temperature of the isotherm may be programmed into the controller and the valve controlled by a feedback loop in order to maintain the desired temperature.

It is to be noted that the apparatus may be able to initiate a response in and/or freeze the tissue sufficiently without actual liquefied gas being sprayed from the catheter, and that a spray of liquid may not be needed if the very cold gas (for example, nitrogen gas at less than 0° C.) can accomplish the task of reducing the temperature of and/or freezing the targeted tissue. Thus, an isotherm of sufficiently low temperature can be created and maintained in proximity with a target tissue for a period of time sufficient to result in initiating a response in the target tissue. As used herein, “in proximity” means sufficiently close to the target tissue to cause a desired reaction in the target tissue, for example, to cause a reduction in the temperature of the target tissue and/or to freeze the target tissue.

In some embodiments, methods of the invention may be used to freeze a target tissue. Freezing is apparent to the physician by the frozen tissue acquiring a white color (sometimes referred to herein as cryofrost), due to surface frost (visible on the monitor 28 in FIG. 3); the white color indicates oral, nasal, pharyngeal and/or laryngeal tissue freezing sufficiently to destroy diseased tissue and/or to initiate a desired response in the tissue. The physician manipulates the naso-laryngoscope 10, vent 42, and/or catheter 20 to treat all of the targeted tissue. Once the operation is complete, the scope 10 with catheter is withdrawn. In embodiments where a guiding device is not required, the catheter may be directly manipulated by the operator to treat all of the targeted tissue and then withdrawn.

The depth of tissue that is frozen can be controlled in three ways. First by the duration of the spray. The second, by the number of freeze/thaw cycles applied. Third, by the amount of area covered by the spray. The depth ranges of the present invention can range from superficial (i.e., epithelium) to deep into the oral, nasal, pharyngeal and/or laryngeal tissue, depending on the desired response and need for tissue injury.

Following freezing, the cells of the treated tissue are damaged or dying. As the treated site heals, the dead cells are typically sloughed off or removed by immune cells. Over time, healthy cells grow in their place to repair the damage and replace injured tissue. Thus, effective cryotherapy can be achieved without gross damage to the oral, nasal, pharyngeal and/or laryngeal tissue (for example, there is no laceration). Typically, there will be no need to further treat the frozen area.

The apparatus shown in FIG. 3 can also be used with the methods of the present invention. Various component of FIG. 3 are more fully described in U.S. Pat. No. 7,025,762 to Johnston et al., which is incorporated by reference. In particular the descriptions of the components described in FIG. 3 are specifically incorporated herein by reference. Other apparatuses capable of delivering liquid cryogen to a catheter, particularly low temperature, low pressure cryogen, may also be employed.

Because the invention uses liquid spray via a catheter 20 rather than contact with a cold solid probe, there is little risk of a cold apparatus sticking to the oral, nasal, pharyngeal and/or laryngeal tissue and tearing the tissue. Even if contact is made between the catheter and the oral, nasal, pharyngeal and/or laryngeal tissue, the plastic material of the catheter, such as TEFLON, is in little danger of sticking to the tissue because of its low thermal conductivity and specific heat. Furthermore, the catheter need not touch the tissue according to many embodiments. In instances where sticking is observed, it may be desirable to equip the guiding device (e.g., endoscope) and/or catheter with a cap or other extension to prevent contact between the catheter and the tissue to be treated.

In embodiments that involve spraying liquid cryogen directly onto tissue, the cooling rate (rate of heat removal) is much higher than with a solid, contact probe because the sprayed liquefied gas can evaporate directly on the target tissue, which absorbs much of the heat of vaporization. The rate of rewarming is also high, since the applied liquid boils away almost instantly. No cold liquid or solid ultimately remains in contact with the tissue, and the depth of freezing can be minimal if desired.

Since freezing is accomplished by boiling liquefied gas (e.g., nitrogen), large volumes of this gas can be generated. This gas can be provided with a mechanism to escape in order to minimize the chance of pressure-related injury. The local pressure can be higher than atmospheric. To minimize any possible chance of a pressure related injury there can be provided several alternative methods for facilitating the evacuation of gas.

A suction tube 41 as seen in FIG. 3, which can run outside of and adjacent to the endoscope or other guiding device 10 and/or catheter 20. Suction may be provided by a suction pump 45 or other conventional suction device.

FIG. 2B shows a catheter tip on the end of the catheter 20 and adapted to spray liquefied gas through one or more openings 49 on catheter 20. When a lateral hole is provided in the wall of the catheter, the distal end of the catheter can be closed so that cryogen is directed laterally. The length of the catheter tip and size and shape of the spray holes can be chosen so that the entire area of the targeted tissue is treated at once without the need for manipulating the endoscope or catheter to treat the targeted area in sequential increments. The catheter tip may be of rigid material such as metal or stiff plastic, preferably the latter. Alternatively, the entire guiding device (e.g., endoscope) and/or catheter may be moved up or down the nasopharyngeal space to ensure that the entire targeted area is sprayed.

FIGS. 2A, 2B, and 2C also show the distal end 12 of the guiding device (e.g., endoscope) 10 including a camera lens 14, illuminating light 16, biopsy channel or lumen 18 with the catheter 20 therein, and an additional lumen 22.

The catheter will have one or more openings 49, whereby cryogen spray exits the catheter and contacts the tissue and/or creates an isotherm in proximity to the target tissue. The openings may be configured in such as way as to allow the cryogen to spray in a substantially perpendicular direction. When used in connection with a spray pattern, the term “substantially perpendicular” is not intended to limit direction of the spray to a plane at an angle of 90 degrees to the axis of the catheter, but includes any type of spray which will allow the targeted tissue of the mouth, tongue, nose, sinuses, pharynx and/or larynx that is coaxial to the catheter to be sprayed, near the locus of the tip of the catheter and to exclude a spray which is only substantially axial.

The end of the catheter 20 may also be cut at an angle to deflect the spray to one side as shown in FIG. 2A. Alternatively, FIG. 2C shows an optional cone-shaped structure 110 disposed around the opening in the catheter to direct the spray to the target tissue.

It is also contemplated that the cryospray may be supplemented with and/or used in conjunction with one or more additives. For example, cryospray may be used as a means of delivering therapeutic agents to the target tissues. Such additives may be mixed with the liquid nitrogen or other cryogen and simultaneously sprayed onto target tissue, or may be delivered (e.g., sprayed or placed on or adjacent to treated tissue) separately from the cryogen before, during or after cryotherapy. Non-limiting examples of contemplated additives include organic chemicals, agents, or compound formulations, inorganic chemicals or agents, gene therapy agents including but not limited to viruses, lipids, other transfection agents or naked circularized or linear DNA, dyes or indicators, either organic or inorganic, gels, liquids, solids, gases and crystals, glues, pharmaceuticals, prodrugs, aerosols, blood, plasma, tissue or other biological products, solvents (covered under chemicals), polymers, plasticizers, and absorbable, expandable materials, nano-technology, robotics, and/or magnetized material/products.

While not wishing to be bound by any particular theory, it is contemplated that delivery of an additive with cryotherapy will facilitate cellular uptake of the additive, especially by cryotreated tissue. In vitro studies investigating the delivery of chemotherapeutic agents to frozen cells demonstrated that cold increases cellular permeability and, thereby, susceptibility to a chemotherapeutic agent that does not otherwise enter cells efficiently. Mir, L M and Rubinsky, B. (2002) Treatment of cancer with cryochemotherapy. Brit J Canc 86, 1658-1660. It is, therefore, contemplated that cryospray-induced cellular permeability may preferentially facilitate the uptake of cryospray additives into treated cells rather than non-target cells.

It is also contemplated that cells that are stimulated to grow and replicate in response to cryotherapy would rapidly assimilate biomaterials from the immediate environment. Thus, cryotherapy may make these cells less selective as to the materials they incorporate and more likely to assimilate cryospray additives. Further, when cells are immediately killed by freezing or sent into apoptosis following exposure, an immune response can be generated. The immune response can include a cytotoxic T cell response, a humoral response or an innate response. The immune response can involve the production of cytokines, chemokines or other signaling molecules and can involve an inflammatory response. It may also stimulate a vaccine-like response. Such mechanisms may modulate the bioavailability or cellular uptake of an additive or the metabolism of a prodrug into its active form.

If gene therapy is used, delivery vectors for gene therapy may include any suitable delivery vector known in the art, such as viruses, liposomes, nanoparticles or naked DNA.

Adenoviruses carrying deletions have been proposed as suitable vehicles for genetic information. Adenoviruses are non-enveloped DNA viruses. Gene-transfer vectors derived from adenoviruses (so-called “adenoviral vectors”) have a number of features that make them particularly useful for gene transfer for such purposes. For example, the biology of the adenovirus has been characterized in detail, the adenovirus is not associated with severe human pathology, the adenovirus is extremely efficient in introducing its DNA into the host cell, the adenovirus can infect a wide variety of cells and has a broad host-range, the adenovirus can be produced in large quantities with relative ease, and the adenovirus can be rendered replication defective by deletions in the early-region 1 (“E1”) of the viral genome.

Non-integrating viruses, such as a cytoplasmic virus, may also be a suitable vector for delivery of genetic material. The genetic material carried by these vectors will thus not be present in the nucleus of the target cell, unless specifically desired. The vector preferably has a low replicative efficiency in the target cell.

Non-lytic viruses, those that will not kill most target cells in the host animal or a tissue culture in a short period of time during which the viable infected cells will be expressing the gene product, may also be used. For example, it preferably will not kill more than about 25% of the target cells it is being used in within 48 hours, more preferably 72 hours, still more preferably, 96 hours. More preferably, it will not kill more than about 10% of the target cells in the host animal or tissue culture it is used in within 48 hours, more preferably 72 hours, and still more preferably 96 hours. Even more preferably, such a transformed target cell population will be expressing the delivered gene product for a period of 1 to 2 weeks after initial infection. This can readily be determined by assaying samples of the target cell for viability, e.g., by staining with trypan blue, and gene expression, e.g., measuring protein production with ELISA.

The term “short-term” delivery system described herein is preferably directed to the use of vector systems that although capable of expressing the desired genetic material for at least about 1 week will result in the transient expression of the gene product. Preferably, the expression will be for less than about 2 months, more preferably, less than about 1 month. In addition, by using an avirulent virus for the selected animal host the virus will not cause disease in the host. If any adverse effects are observed, such effects can be further curtailed as described below. Moreover, the delivery system described herein is capable of “controlled release” of a desired protein or other gene product by continuously expressing specific amounts of the protein over a given period of time.

Suitable non-integrating viruses are cytoplasmic viruses. These include both DNA and RNA viruses. DNA viruses include poxviruses such as suipox (e.g. swine pox) capripox, leporipox, avipox (e.g. fowl pox, canary pox) and orthopox (e.g. ectomelia, rabbit pox). Other DNA viruses include iridoviruses such as various insect and frog viruses.

RNA viruses include retroviruses (e.g., lentiviruses) picornaviruses, caliciviruses, togaviruses, rhaboviruses and coronaviruses. Picornaviruses include enterovirus, cardiovirus, rhinovirus, apthovirus, and hepatitis A. Calicivirus include vesicular exanthema virus of swine, dogs or mink, feline calicivirus and caliciviruses of calves, swine, dogs, fowl and chimps. Togaviruses include bovine viral diarrhea virus, hog cholera, and border disease of sheep. Rhabdoviruses include vesiculoviruses such as vesicular stomatitis virus and Lyssaviruses such as rabies. Coronaviruses include infectious bronchitis virus of fowl, transmissible gastroenteritis virus of swine, hemagglutinin encephalyomyelitis virus of swine, turkey, bluecomb virus, calf coronavirus and feline infectious peritonitis virus.

DNA viruses may be preferred for use as vectors. For example, pox viruses are well known cytoplasmic viruses. Thus, genetic material expressed by such viral vectors typically remain in the cytoplasm and do not have the potential for inadvertent integration of the genetic material carried into host cell genes, unless specific steps are taken such as described above. Furthermore, because these vectors have a large genome, they can readily be used to deliver a wide range of genetic material including multiple genes (i.e., act as a multivalent vector).

The viral vectors may be oncolytic viral vectors. Oncolytic viral vectors are viral vectors which selectively replicate in tumor cells and destroy the cells in which they replicate, but do not replicate to any significant degree, in non-tumor cells. For example, oncolytic adenoviral vector may have a tissue-specific transcriptional regulatory sequence is operably linked to said gene essential for replication as described above. Alternatively, oncolytic adenoviral particles may include a mutation in a gene essential for adenoviral replication, such as the E1a or E1b genes. Such mutations may render adenoviral replication specific for tumor tissue, e.g. if the cells of said tissue have a defect in the p53 or Rb pathways. Oncolytic adenoviral vectors may or may not include a heterologous gene in addition to the adenoviral elements necessary for replication.

In a further embodiment, the present invention provides vector constructs which include a therapeutic gene. A therapeutic gene can be one that exerts its effect at the level of RNA or protein. For instance, a protein encoded by a therapeutic gene can be employed in the treatment of an inherited disease, e.g., the use of a cDNA encoding the cystic fibrosis transmembrane conductance regulator in the treatment of cystic fibrosis. The protein encoded by the therapeutic gene can exert its therapeutic effect by causing cell death. For instance, expression of the protein, itself, can lead to cell death, as with expression of diphtheria toxin A, or the expression of the protein can render cells selectively sensitive to certain drugs, e.g., expression of the Herpes simplex (HSV) thymidine kinase gene renders cells sensitive to antiviral compounds, such as acyclovir, gancyclovir and FIAU (1-(2-deoxy-2-fluoro-β-D-arabinofuranosil)-5-iodouracil). Alternatively, the therapeutic gene can exert its effect at the level of RNA, for instance, by encoding an antisense message or ribozyme, a protein that affects splicing or 3′ processing (e.g., polyadenylation), or a protein that affects the level of expression of another gene within the cell, e.g. by mediating an altered rate of mRNA accumulation, an alteration of mRNA transport, and/or a change in post-transcriptional regulation.

Tumor suppressor genes are genes that, in their wild-type alleles, express proteins that suppress abnormal cellular proliferation and may also be delivered or upregulated as part of cryotherapy. When the gene coding for a tumor suppressor protein is mutated or deleted, the resulting mutant protein or the complete lack of tumor suppressor protein expression may fail to correctly regulate cellular proliferation, and abnormal cellular proliferation may take place, particularly if there is already existing damage to the cellular regulatory mechanism. A number of well-studied human tumors and tumor cell lines have been shown to have missing or nonfunctional tumor suppressor genes. Examples of tumor suppression genes include, but are not limited to, the retinoblastoma susceptibility gene or RB gene, the p53 gene, the deleted in colon carcinoma (DCC) gene and the neurofibromatosis type 1 (NF-1) tumor suppressor gene (Weinberg, R. A. Science, 1991, 254:1138-1146). Loss of function or inactivation of tumor suppressor genes may play a central role in the initiation and/or progression of a significant number of human cancers.

For human patients, the therapeutic gene will generally be of human origin although genes of closely related species that exhibit high homology and biologically identical or equivalent function in humans may be used if the gene does not produce an adverse immune reaction in the recipient. As used herein, the term “high homology” refers to genes that have 85% or more identical base pairs, preferably at least 90%, more preferably at least 95% and most preferably at least 99%. A therapeutically effective amount of a nucleic acid sequence or a therapeutic gene is an amount effective at dosages and for a period of time necessary to achieve the desired result. This amount may vary according to various factors, including but not limited to sex, age, weight of a subject, and the like.

The DNA sequence encoding at least one therapeutic gene is under the control of a suitable promoter. Suitable promoters which may be employed include, but are not limited to, adenoviral promoters, such as the adenoviral major late promoter; or hetorologous promoters, such as the cytomegalovirus (CMV) promoter; the Rous Sarcoma Virus (RSV) promoter; inducible promoters, such as the MMT promoter, the metallothionein promoter; heat shock promoters; the albumin promoter; and the ApoAI promoter. In a preferred embodiment, the promoter of the invention is an E2F-responsive promoter, in particular the E2F-1 promoter. In one embodiment of this invention, the E2F promoter is operatively linked to the E1a gene.

In addition to the E2F promoter, use of the following tumor selective promoters are contemplated: osteocalcin, L-plastin, CEA, AVP, c-myc, telomerase, skp-2, psma, cyclin A, and cdc25 promoters. It is to be understood, however, that the scope of the present invention is not to be limited to specific foreign genes or promoters. The selection of a particular promoter and/or enhancer depends on what cell type is to be used to express the protein of interest. Some eukaryotic promoters and enhancers have a broad host range while others are functional in a limited subset of cell types.

The liposome compositions can provide highly efficient delivery of biologically active agents to cells. Liposome vesicles can be prepared from a mixture of a cationic lipopolyamine and a neutral lipid and form a bi- or multilamellar membrane structure (referred to herein as “DLS-liposomes”). For example, one may use a spermine-5-carboxy-glycinedioctadecylamide (referred to herein as “DOGS”) as the cationic lipopolyamine and dioleylphosphatidyl ethanolamine (referred to herein as “DOPE”) as the neutral lipid. Other liposome compositions can also be used. Use of such liposomal vehicles make possible high transfection efficiency of biologically active materials into cells.

The presence of at least one neutral lipid in combination with at least one cationic lipopolyamine makes possible the formation of liposomes after hydration. Liposomes may be prepared by mixing together each of a cationic lipopolyamine and a neutral lipid in a molar ratio ranging from, for example, a ratio of 0.02:1 to a ratio of 2:1; evaporating the mixture to dryness; and rehydrating. In order to introduce a biologically active agent into the liposomes, such agent can be added prior to or after rehydration of the dried film.

Nucleic acids may be associated with the liposomes. This association may be accomplished in at least two ways: (1) complex formation between the cationic liposome vesicle and negatively charged polyanion, such as nucleic acid or (2) encapsulation in the cationic liposome vesicle. Such a formulation may have applications for treating subjects via effective delivery of oligonucleotides or gene-expressing nucleic acid vectors (e.g. plasmids or viral vectors) into cells. Therefore, such a method of drug delivery is useful for the transport of nucleic acid based therapeutics.

It is also contemplated that cryotherapy may be utilized to manipulate immune system responses in oral, nasal, pharyngeal and/or laryngeal tissue. While not wishing to be bound by any particular theory, it is contemplated that cells critically damaged by cryospray will initiate their apoptotic machinery. These dead and dying cells may recruit immune effecter cells, such as macrophages or other phagocytes and T helper cells, to the treated site.

By taking advantage of this mechanism, it is contemplated that cryotherapy may be used to initiate a targeted immune response in oral, nasal, pharyngeal and/or laryngeal tissue for the treatment of a disease. Recruiting immune cells to a site of pathology may increase the likelihood of encounter and, thus, allow the immune system to recognize a tumor cell, pathogen, or other cells that may otherwise evade the normal innate or adaptive immune system responses. Such methods may be used to treat cancer in oral, nasal, pharyngeal and/or laryngeal tissue, infections in oral, nasal, pharyngeal and/or laryngeal tissue, or other conditions that may benefit from an increased or targeted immune response. Cryotherapy may also be used to stimulate a vaccine-like response. An inflammatory response associated with cryogen application may also beneficially effect the desired therapy. For example, inflamed tissue can be more permeable to therapeutic agents than non-inflamed tissue.

It is also contemplated that cryotherapy may be used to suppress inflammation as well as to induce a systemic immune and antimetastatic response. Cryotherapy is frequently used to treat and alleviate inflammation of other parts of the body as well as to induce a systemic immune and antimetastatic response, such as by application of ice packs to injured muscle tissue. While not wishing to be bound by any particular theory, it is contemplated that cryospray therapy may be used to cool target oral, nasal, pharyngeal and/or laryngeal tissue without freezing and concomitant cellular damage and/or death. Alternatively, more intense cryotherapy may be used to initiate a response in and/or freeze and kill nerve endings that are sending pain signals, thereby inducing an analgesic effect. Such cryo treatment may alleviate swelling, heat, and pain of oral, nasal, pharyngeal and/or laryngeal tissue caused by inflammation. Such methods may be used to treat allergic conditions and/or inflammatory conditions, for example, allergic rhinitis, oral ulcers, stomatitis or gingival hyperplasia, laryngitis, or granulomas as well as other conditions.

In a further contemplated embodiment, it is envisioned that cryotherapy may be used to stimulate chondrogenesis. Cartilage that has been damaged due to physical injury, chronic inflammation, or any other cause may be treated with cryospray. The regeneration of cartilage has been observed after cryotherapy in lung tissue (see United States provisional application for patent Ser. No. 60/992,580 filed Dec. 5, 2007 the entire contents of which are specifically incorporated herein by reference).

The cryogen spray can be conducted in such a manner as to allow constant direct visualization by the physician of the targeted tissue treatment as it occurs. Condensation on the lens of the guiding device (e.g., endoscope) may be avoided by means of the suction pump, which will immediately suck out the moist air which is present prior to the arrival of the liquid spray or cold gas. This condensation effect is augmented by the fact that the catheter itself may not be wrapped in additional insulation. Fog or frost can also be prevented by flushing out the area around the target tissue with the liquefied gas, which is extremely dry.

An electronic monitoring and recording system may also be used with the apparatus during treatment of oral, nasal, pharyngeal and/or laryngeal tissue and is described in U.S. Pat. No. 7,025,762. The electronic components of the system may comprise a control box, temperature sensor or probe and timer. Also connected to the monitoring and recording system may be a foot-pedal for actuating a solenoid-controlled valve and a recording console. An electric power cord can run from solenoid to the control box. The electronic monitoring and recording system may record the times at which spraying of cryogen starts and ends. Temperature in the treatment space may also be recorded for the cryosurgery at pre-selected time increments. This recordation allows for better data acquisition and documentation. The electronic console can be preprogrammed to be patient specific.

The components or paraphernalia required to practice the method of the present invention may be packaged and sold or otherwise provided to health-care providers in the form of a kit. The kit is preferably sealed in a sterile manner for opening at the site of the procedure. The kit can include the catheter, having a spray nozzle at one end, as well as a connector for connecting the catheter to the source of liquefied gas. This connector may be a simple luer connection on the opposite end of the catheter from the spray nozzle. Any connector known to those skilled in the art may be used to allow the catheter to be connected to the gas source.

One example of suitable steps for performing the methods of the invention is as follows. A cryogen source is provided. The proximal end of a suitable catheter is attached to the cryogen source so as to be in fluid communication therewith once the source is activated. A suction tube, attached at a proximal end to a suction device can be inserted such that the distal end of the suction tube is near the target tissue or otherwise in fluid communication with the treatment space surrounding the tissue. The distal end of the suction tube can be positioned proximal to the target tissue so as not to interfere with the treatment. If suction is to be performed through an endoscope or not performed, the suction tube can be omitted. An endoscope can be inserted into the patient such that the distal end of the scope is near the target tissue and the tissue is visualized. The endoscope can be supplied with light and a fiberoptic visualization system or television camera. Optionally, attached to the endoscope will be a temperature probe to sense the temperature and report the temperature to the recording console, or a temperature sensor can be placed through a lumen of the endoscope. The distal end of the catheter can then be inserted through the working channel (lumen) of the endoscope. In the event that the distal end of the catheter includes a directional tip that does not fit in the lumen, it is possible to thread the proximal end of the catheter through the endoscope and connect the proximal end to the cryogen source after it has been inserted. The distal tip of the catheter can be positioned near the tissue to be treated, with the spray tip (open distal end or lateral hole) directed at the tissue. The treatment space can be vented using the suction tube to remove moist air (if required). Tissue can be treated by spraying cryogen at low pressure and low temperature. Cryogen will come from the tip of the catheter. A single cycle of cryospray can last for about 10 seconds to about 2 minutes. Shorter or longer times may be appropriate depending on the size and nature of the tissue to be treated. Any number of cryospray cycles may be performed. The tissue can be visualized between cryospray cycles and/or when the treatment is complete to ensure adequate tissue response has occurred and treatment repeated if necessary. Once the desired response has been achieved, the endoscope and suction tube can be removed. Similar methods may be performed without the use of a guiding device (e.g., endoscope) where the tissue to be treated can be directly accessed with the catheter without the need for a guiding device.

In some embodiments, the desired response may be to freeze a target tissue to a desired depth. The preliminary test results indicate that a 5 second “cryofrost” time over varying cycles was adequate to ensure the appropriate tissue destruction, and thus appropriate cellular healing of damaged tissue for many applications. “Cryofrost” is a term defined by the instance that the normally “pinkish” targeted tissue turns white. A range for the “cryofrost” time could be about 5-10 seconds to about 2 minutes or more depending on the substrate to be treated.

Due to the nature of the system, “cryofrost” may not immediately occur, but may require that the fitting and catheter system become cool so that cryogen being sprayed from the distal end of the catheter is adequately cold to effect the cryofrost. This can require approximately 20-30 seconds from the time that the cryogen begins to flow. Of course, this time may be longer or shorter depending on the temperature of the cryogen, the length of the flow path, the materials from which the system is constructed and environmental conditions.

During animal testing the approximate temperature that cryofrost was first observed was at approximately −10° C. The temperature range for cryofrost would be approximately −10 to −90° C.

Cryotherapy may be useful in treating, preventing, or curing diseases of oral, nasal, pharyngeal and/or laryngeal tissue such as, but not limited to, benign or malignant tumors, lesions, dysplastic tissue, and neoplastic diseases, infectious diseases, and a variety of conditions characterized by inflammation of oral, nasal, pharyngeal and/or laryngeal tissue as well as to induce a systemic immune and antimetastatic response. Additionally, it may be used to remove any unnecessary or exuberant tissue from this area as well.

Neoplastic or dysplastic diseases of oral, nasal, pharyngeal and/or laryngeal tissue

It is contemplated that cryotherapy may be used for treating forms of neoplastic and dysplastic diseases such as, but not limited to, cancers of the mouth, nose, sinuses, salivary glands, throat and lymph nodes in the neck. Any type of cancer (e.g., squamous cell carcinoma) can be treated using the methods of the invention. Cancerous or dysplastic tissue may be identified using any technique known in the art, for example, endoscopic examination, biopsy etc. Cancerous or dysplastic tissue may then be sprayed with cryogen and/or brought into proximity with an isotherm for a period of time sufficient to freeze the cancerous material and, optionally, a margin of healthy tissue surrounding the cancerous tissue.

Additionally, as some cancer cells may survive cryotherapy and cause cancer recurrence, it is further contemplated that one or more anti-cancer agents may be applied to the treated area, for example, antineoplastic agents and/or gene therapy may be used in combination with the cryo procedure. For example, tumor suppressor genes or genes that promote apoptosis of the cancer cells may be administered.

In some embodiments, materials and methods of the invention may be used in conjunction with standard surgical techniques to treat cancer. For example, a tumor may be removed using standard surgery and the margins of the surgical site may be treated with cryospray in order to clear and/or enhance the margins.

Infections of oral, nasal, pharyngeal and/or laryngeal tissue

Infections may be caused by any pathogenic organism, such as bacteria, fungi, viruses, or parasites. While not wishing to be bound by any particular theory, it is contemplated that cryotherapy may be used to kill pathogens by freezing them and/or activating a cold shock response that inhibits growth and pathogenesis. It is further contemplated that cryotherapy may also be used to stimulate an innate, humoral, and/or cell-mediated immune response, thereby signaling immune effecter cells to respond and fight the source of infection. Infections may lead to chronic inflammation (e.g., sinusitis). Cryogen can be used to dampen the inflammatory response as well as for direct insult to the offending pathogen thus restoring the appropriate host response and establishing control of the infectious agent.

In a particular embodiment, materials and methods of the invention can be used to treat chronically infected tonsils. Tonsils may be sprayed with cryogen and/or tonsils may be brought in proximity to an isotherm for a period of time sufficient to initiate a response.

In another embodiment, materials and methods of the invention can be used to treat chronic or recurrent infection of the adenoids. Adenoids may be sprayed with cryogen and/or adenoids may be brought in proximity to an isotherm for a period of time sufficient to initiate a response.

In another specific embodiment, materials and methods of the invention may be used to treat bacterial infections, in particular, streptococcal infections. Strep throat is caused by Group A Streptococcus bacteria. It is the most common bacterial infection of the throat and may be treated used the materials and methods of the invention.

Removal of unwanted oral, nasal, pharyngeal and/or laryngeal tissue

Materials and Methods of the invention may be used to remove unwanted tissue. Typically, the unwanted tissue is sprayed with cryogen and/or maintained in proximity to an isotherm for a period of time sufficient to freeze the tissue.

In a particular embodiment, materials and methods of the invention can be used to remove tonsils. Tonsils may be sprayed with cryogen and/or tonsils may be brought in proximity to an isotherm for a period of time sufficient to freeze all or a portion of the tonsil tissue.

In other specific embodiments, unwanted tissue may comprise adenoidal tissue or leukoplakia. Adenoid removal is surgery to take out the adenoid glands, which are located between the nasal airway and the back of the throat (nasopharynx). All or a portion of the adenoids may be removed or as a result of treatment, may atrophy or shrink. Treatment of leukoplakia will restore normal mucosa and therefore reduce the risk of developing a carcinoma.

In some embodiments, materials and methods of the invention may be used in conjunction with standard surgical techniques to remove unwanted tissue. For example, unwanted tissue may be removed using standard surgery and the margins of the surgical site may be treated with cryospray in order to clear and/or enhance the margins.

Materials and Methods of the invention will be used to treat oral, nasal, pharyngeal and/or laryngeal tissue. Target tissue (e.g., tonsils, adenoids, lesions etc) will be identified and sprayed with cryogen and/or maintained in proximity to an isotherm. Optionally, target tissue may be treated a plurality of times, for example, 2, 3, 4, 5, 6 times until a desired level of cryofrost may be observed.

### Example 1

### Materials and Methods

Any device adapted to deliver cryogen can be used to practice the invention as described herein. For example, in the method described below, spray cryotherapy was performed with the CryoSpray Ablation™ System (“CSA” System, Model CC2-NAM, CSA Medical, Inc) which has 510(k) clearance by the U.S. Food and Drug Administration and CE mark for use in Europe [as a cryosurgical tool in the fields of general surgery, specifically for endoscopic applications]. The CSA System, a non-contact method of cryotherapy, was used to apply medical-grade liquid nitrogen (196° C.), directly to the tissue via a low-pressure, disposable 7 French cryocatheter introduced through the vocal cords through the working channel of a therapeutic flexible bronchoscope [(Olympus BF-X1T160 or BF-X1T180)]. A waiver was obtained from the Medstar Institutional Review Board (Hyattsville, Md.) such that the data might be reviewed. Patients initially received 4 cycles of 5 second spray cryotherapy with a complete thaw of the treated area between each application. If needed, balloon dilation followed using appropriately sized balloons (CRE balloon, Boston Scientific, Natick, Mass.) with subsequent delivery of an additional 2 cycles of 5 second spray cryotherapy after the mechanical injury. Freeze and thaw techniques were monitored by direct visualization. The duration and extent of the cryogen spray to the selected site was at all times under the control of the physician.

Tracheal stenosis of unclear etiology:

A 43 year old Caucasian woman with history of gastroesophageal reflux, chronic allergic rhinitis, and no history of smoking presented with stridor, hoarseness and trouble breathing. The dyspnea and stridor had progressively worsened over the preceding 24 months. She received a Speech, Language, Pathology evaluation after she developed hoarseness and a change in voice quality. She had carried the questionable diagnosis of severe asthma, which was diagnosed roughly 2 to 3 years prior to the development of her hoarseness and was treated with increasing amounts of oral corticosteroids with marginal control of her symptoms.

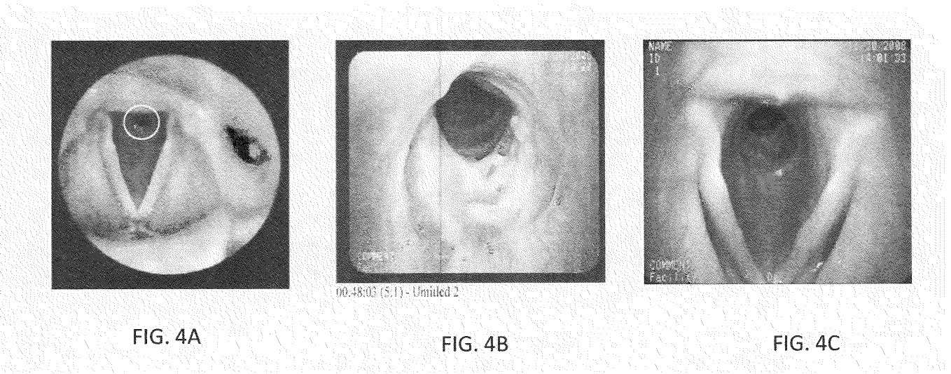

Flexible laryngoscopy was performed and revealed a subglottic stricture beginning at the distal end of the thyroid cartilage and extending down to the second tracheal ring. The lumen of the proximal trachea measured 5-6 mm in diameter (FIG. 4A). Work up for the etiology of the subglottic stricture was unrevealing. The patient was treated with 4 cycles of 5 second spray cryotherapy, with a complete interim thaw of the treated tissue, lasting approximately 30 to 40 seconds between sprays. This was followed by dilatation with an 8-9-10 mm CRE balloon and repeat dilation with a 12-13.5-15 mm CRE balloon.

Spray cryotherapy was then delivered to the dilated wound but with 2 cycles of 5 second sprays in an attempt to modify the injury response. The patient tolerated the procedure well, and no adverse events occurred. This treatment led to complete remission of the patient's hoarseness and profound improvement in her breathlessness within seven days (FIG. 4B shows 10 days post-treatment). Follow-up at 1 (FIG. 4c), 3, and 6 months post-treatment confirmed the patient had near complete luminal patency of the airway without evidence of restricturing or granulation tissue. She remains symptom-free and no longer requires use of corticosteroids.

### Example 2

Glottic stricture and vocal cord stenosis following radiation:

The following method was performed using the materials and methods described above in Example 1.

A 74 year old Caucasian female with a 53 year history of smoking and gastroesophageal reflux disease had recently completed 28 of 33 sessions of radiation therapy for a recent diagnosis of squamous cell carcinoma of the right vocal cord. Over the preceding month, she had developed progressive severe dyspnea, stridor and hoarseness.

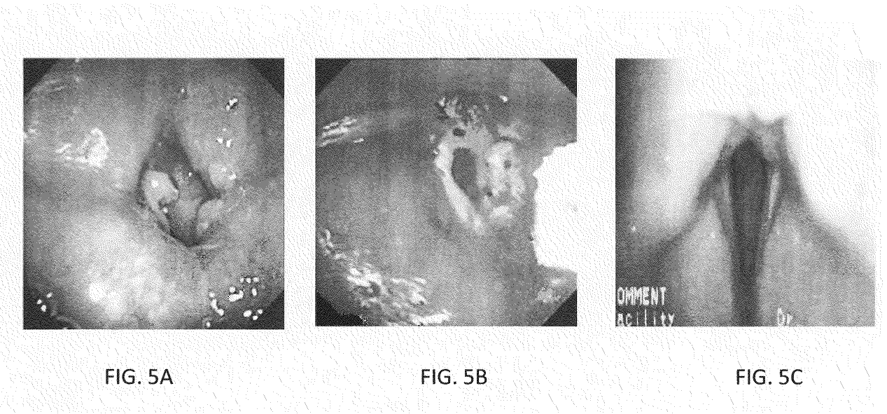

A month prior to presentation, when the symptoms began, she had received a solumedrol taper for her shortness of breath along with a V/Q scan that was negative for pulmonary embolism. Four weeks later, evaluation with fiberoptic laryngoscopy by Speech, Language, Pathology revealed a web-like circumferential occlusion of the glottic opening with a 4 to 5 mm aperture likely a consequence of her radiation therapy. A bronchoscopy and suspension microlaryngoscopy confirmed laryngeal stenosis secondary to web formation from the mid-cord to the anterior commisure (FIG. 5A). The patient was treated with 4 cycles of 5 seconds of spray cryotherapy. This was followed by dilatation with an 8-9-10 mm CRE balloon, then further dilatation with a 12-13.5-15 mm CRE balloon. A subsequent 2 cycles of 5 second spray cryotherapy was delivered. Airway patency was achieved with an increased post-procedure lumen to approximately 12 to 15 mm in diameter. At 12 weeks post procedure, repeat Speech, Language, Pathology evaluation along with flexible laryngoscopy demonstrated a normal appearance of her vocal cords and larynx without any return of her previous symptoms. In addition, pulmonary function tests demonstrated an increase in her peak flow rates of nearly 180% (1.82 L/min to 5.03 L/min).

### Example 3

Tracheobronchiomalacia with partial obstruction of previous tracheostomy tube:

The following method was performed using the materials and methods described above in Example 1.

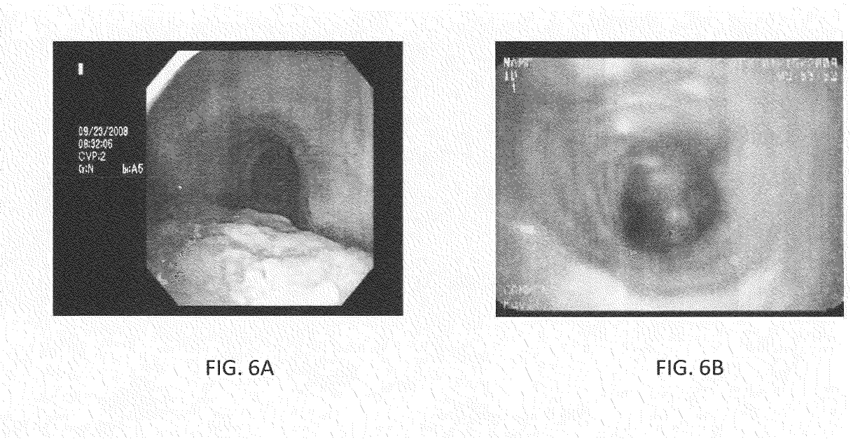

A 33 year old Caucasian female with a 20 year history of smoking and asthma since childhood presented with progressive dyspnea on exertion, recent exacerbations of chronic breathlessness, and an occasional cough that was negative for hemoptysis. CT scan demonstrated the congenital absence of the left lobe of the thyroid and a prominent right lobe. Bronchoscopy revealed significant tracheobronchiomalacia involving the lateral and posterior wall extending from the trachea to the proximal left mainstem bronchus, which further work-up deemed to be idiopathic in nature (FIG. 6). Because of her worsening dyspnea, tracheal stenting with a silicone stent was attempted. While initially tolerated well, roughly 9 months later she was found to have developed extensive granulation tissue at both the proximal and distal ends of the stent resulting in obstruction and dyspnea. This was removed and temporized with a metal hybrid stent which ultimately fractured and migrated.

Following removal of the stent, the patient underwent a tracheostomy followed by Ttube placement to increase airway patency. Six months later she developed a stricture and granulation tissue in the subglottic region, and at the distal end of the T-tube in the mid trachea. The patient then underwent treatment with spray cryotherapy and received 4 cycles of 5 seconds of spray cryotherapy at both the sites as well as at the ostomy site which also had developed granulation tissue. Follow-up was conducted regularly and now at 9 months post treatment, airway examination confirmed no recurrence of the stricture and no granulation tissue with the t-tube remaining in place.

This case series represents the first use of low pressure spray cryotherapy to treat glottic and subglottic stenosis in three patients in whom standard surgical and endoluminal treatment modalities either previously failed or were not suitable. Spray cryotherapy with or without balloon dilatation allows for immediate removal of membranous and/or fibrotic strictures and granulation tissue both immediately and at 9 months of follow-up. Bleeding is minimized and there is at least partial restoration of normal mucosa on follow up examinations. Patients all achieved improvement in their airway patency as well as restoration of other laryngeal functions such as phonation. These cases demonstrate that spray cryotherapy can be used in management and treatment of subglottic stenosis regardless of the etiology. These cases also demonstrate that spray cryotherapy is effective as an adjunctive therapy when following surgical resection or as a temporizing measure when standard surgical or endoluminal approaches have either failed or are not feasible.

While the invention has been described in detail, and with reference to specific embodiments thereof, it will be apparent to one of ordinary skill in the art that various changes and modifications can be made therein without departing from the spirit and scope thereof and such changes and modifications may be practiced within the scope of the appended claims. All patents and publications herein are incorporated by reference to the same extent as if each individual publication was specifically and individually indicated to be incorporated by reference in their entirety.

What is claimed is:

1. A method for treating oral, nasal, pharyngeal and/or laryngeal tissue, comprising:
spraying the tissue with a cryogen, or using the cryogen to create an isotherm in proximity to the tissue.

2. A method according to claim 1, wherein the cryogen is a liquefied gas.

3. A method according to claim 2, wherein the liquid gas is selected from the group consisting of oxygen, a nitrogen oxide, nitrogen and argon.

4. A method according to claim 1, wherein the cryogen is carbon dioxide.

5. A method according to claim 1, wherein the tissue is sprayed with cryogen or is in proximity to the isotherm for a period of time sufficient to initiate a tissue response.

6. A method according to claim 1, wherein a proximal end of a catheter is connected to a cryogen source and a distal end of the catheter is placed in proximity to the tissue and cryogen flows from the source through the distal end to the tissue.

7. A method according to claim 6, wherein the distal end of the catheter is guided to the tissue using a guiding device.

8. A method according to claim 7, wherein the guiding device comprises a video camera and the distal end of the guiding device and/or the catheter is guided to the tissue by observing the distal end of the catheter and/or guiding device on a video monitor.

9. A method according to claim 1, wherein the tissue comprises unwanted tissue.

10. A method according to claim 9, wherein the unwanted tissue comprises cancerous cells.

11. A method according to claim 1, wherein the tissue is sprayed with cryogen or is maintained in proximity to the isotherm for a period of time sufficient to freeze the tissue.

12. A method for treating a lesion in oral, nasal, pharyngeal and/or laryngeal tissue, comprising:
spraying tissue comprising a lesion with a cryogen or using the cryogen to create an isotherm in proximity to the tissue.

13. A method according to claim 12, wherein the cryogen is a liquefied gas.

14. A method according to claim 13, wherein the liquid gas is selected from the group consisting of oxygen, a nitrogen oxide, nitrogen and argon.

15. A method according to claim 12, wherein the cryogen is carbon dioxide.

16. A method according to claim 12, wherein the tissue is sprayed with cryogen or is in proximity to the isotherm for a period of time sufficient to initiate a tissue response.

17. A method according to claim 12, wherein a proximal end of a catheter is connected to a cryogen source and a distal end of the catheter is placed in proximity to the tissue and cryogen flows from the source through the distal end to the tissue.

18. A method according to claim 17, wherein the distal end of the catheter is guided to the tissue using a guiding device.

19. A method according to claim 18, wherein the guiding device comprises a video camera and the distal end of the guiding device and/or the catheter is guided to the tissue by observing the distal end of the catheter and/or guiding device on a video monitor.

20. A method according to claim 12, wherein the lesion comprises unwanted tissue.

21. A method according to claim 20, wherein the unwanted tissue comprises cancerous cells.

22. A method according to claim 12, wherein the tissue is sprayed with cryogen or is maintained in proximity to the isotherm for a period of time sufficient to freeze the tissue.

23. A method for stimulating a response in oral, nasal, pharyngeal and/or laryngeal tissue, comprising:
spraying the tissue with a cryogen or using the cryogen to create an isotherm in proximity to the tissue.

24. A method according to claim 23, wherein the cryogen is a liquid gas.

25. A method according to claim 24, wherein the liquid gas is selected from the group consisting of oxygen, a nitrogen oxide, nitrogen and argon.

26. A method according to claim 23, wherein the cryogen is carbon dioxide.

27. A method according to claim 23, wherein the tissue is sprayed with cryogen or is in proximity to the isotherm for a period of time sufficient to initiate a tissue response.

28. A method according to claim 23, wherein a proximal end of a catheter is connected to a cryogen source and a distal end of the catheter is placed in proximity to the tissue and cryogen flows from the source through the distal end to the tissue.

29. A method according to claim 28, wherein the distal end of the catheter is guided to the tissue using a guiding device.

30. A method according to claim 29, wherein the guiding device comprises a video camera and the distal end of the guiding device and/or the catheter is guided to the tissue by observing the distal end of the catheter and/or guiding device on a video monitor.

31. A method according to claim 23, wherein the tissue comprises a lesion.

32. A method according to claim 23, wherein the tissue comprises unwanted tissue.

33. A method according to claim 32, wherein the unwanted tissue comprises cancerous cells.

34. A method according to claim 23, wherein the tissue is sprayed with cryogen or is maintained in proximity to the isotherm for a period of time sufficient to freeze the tissue.

35. A method of treating an infection in oral, nasal, pharyngeal and/or laryngeal tissue, comprising:
spraying an infected oral, nasal, pharyngeal and/or laryngeal tissue with a cryogen or using the cryogen to create an isotherm in proximity to the tissue.

36. A method according to claim 35, wherein the cryogen is a liquid gas.

37. A method according to claim 36, wherein the liquid gas is selected from the group consisting of oxygen, a nitrogen oxide, nitrogen and argon.

38. A method according to claim 35, wherein the cryogen is carbon dioxide.

39. A method according to claim 35, wherein the tissue is sprayed with cryogen or is in proximity to the isotherm for a period of time sufficient to initiate a tissue response.

40. A method according to claim 35, wherein a proximal end of a catheter is connected to a cryogen source and a distal end of the catheter is placed in proximity to the tissue and cryogen flows from the source through the distal end to the tissue.

41. A method according to claim 40, wherein the distal end of the catheter is guided to the tissue using a guiding device.

42. A method according to claim 41, wherein the guiding device comprises a video camera and the distal end of the guiding device and/or the catheter is guided to the tissue by observing the distal end of the catheter and/or guiding device on a video monitor.

43. A method according to claim 35, wherein the tissue comprises a lesion.

44. A method according to claim 35, wherein the tissue comprises unwanted tissue.

45. A method according to claim 44, wherein the unwanted tissue comprises cancerous cells.

46. A method according to claim 35, wherein the tissue is sprayed with cryogen or is maintained in proximity to the isotherm for a period of time sufficient to freeze the tissue.

47. A method of treating unwanted oral, nasal, pharyngeal and/or laryngeal tissue, comprising:
spraying unwanted tissue with a cryogen or using the cryogen to create an isotherm in proximity to the tissue.

48. A method according to claim 47, wherein the cryogen is a liquid gas.

49. A method according to claim 48, wherein the liquid gas is selected from the group consisting of oxygen, a nitrogen oxide, nitrogen and argon.

50. A method according to claim 47, wherein the cryogen is carbon dioxide.

51. A method according to claim 47, wherein the tissue is sprayed with cryogen or is in proximity to the isotherm for a period of time sufficient to initiate a tissue response.

52. A method according to claim 47, wherein a proximal end of a catheter is connected to a cryogen source and a distal end of the catheter is placed in proximity to the tissue and cryogen flows from the source through the distal end to the tissue.

53. A method according to claim 52, wherein the distal end of the catheter is guided to the tissue using a guiding device.

54. A method according to claim 53, wherein the guiding device comprises a video camera and the distal end of the guiding device and/or the catheter is guided to the tissue by observing the distal end of the catheter and/or guiding device on a video monitor.

55. A method according to claim 47, wherein the tissue comprises a lesion.

56. A method according to claim 47, wherein the unwanted tissue comprises cancerous cells.

57. A method according to claim 47, wherein the tissue is sprayed with cryogen or is maintained in proximity to the isotherm for a period of time sufficient to freeze the tissue.

58. A method of modulating an immune response in oral, nasal, pharyngeal and/or laryngeal tissue, comprising:
spraying the tissue with cryogen or using the cryogen to create an isotherm in proximity to the tissue.

59. A method according to claim 58, wherein the cryogen is a liquid gas.

60. A method according to claim 59, wherein the liquid gas is selected from the group consisting of oxygen, a nitrogen oxide, nitrogen and argon.

61. A method according to claim 58, wherein the cryogen is carbon dioxide.

62. A method according to claim 58, wherein a proximal end of a catheter is connected to a cryogen source and a distal end of the catheter is placed in proximity to the tissue and cryogen flows from the source through the distal end to the tissue.

63. A method according to claim 63, wherein the distal end of the catheter is guided to the tissue using a guiding device.

64. A method according to claim 64, wherein the guiding device comprises a video camera and the distal end of the guiding device and/or the catheter is guided to the tissue by observing the distal end of the catheter and/or guiding device on a video monitor.

65. A method according to claim 58, wherein the immune response is reduced.

66. A method according to claim 58, wherein the immune response is increased.

67. A method according to claim 58, wherein the tissue is sprayed with cryogen or is maintained in proximity to the isotherm for a period of time sufficient to freeze the tissue.

68. A method of treating benign or malignant tumors or lesions, dysplastic tissue, and/or neoplastic tissue or disease in a subject in need thereof, comprising:
spraying oral, nasal, pharyngeal and/or laryngeal tissue in the subject with a cryogen, or using the cryogen to create an isotherm in proximity to oral, nasal, pharyngeal and/or laryngeal tissue, wherein the oral, nasal, pharyngeal and/or laryngeal tissue comprises the benign or malignant tumors or lesions, dysplastic tissue, and/or neoplastic tissue or disease tissue.

69. A method according to claim 68, wherein the cryogen is a liquid gas.

70. A method according to claim 69, wherein the liquid gas is selected from the group consisting of oxygen, a nitrogen oxide, nitrogen and argon.

71. A method according to claim 68, wherein the cryogen is carbon dioxide.

72. A method according to claim 68, wherein the tissue is sprayed with cryogen or is in proximity to the isotherm for a period of time sufficient to initiate a tissue response.

73. A method according to claim 68, wherein a proximal end of a catheter is connected to a cryogen source and a distal end of the catheter is placed in proximity to the tissue and cryogen flows from the source through the distal end to the tissue.

74. A method according to claim 73, wherein the distal end of the catheter is guided to the tissue using a guiding device.

75. A method according to claim 74, wherein the guiding device comprises a video camera and the distal end of the guiding device and/or the catheter is guided to the tissue by observing the distal end of the catheter and/or guiding device on a video monitor.

76. A method according to claim 68, wherein the tissue is sprayed with cryogen or is maintained in proximity to the isotherm for a period of time sufficient to freeze the tissue.

77. A cryotherapy system adapted for spraying oral, nasal, pharyngeal and/or laryngeal tissue in the subject with a cryogen, or to create an isotherm in proximity to oral, nasal, pharyngeal and/or laryngeal tissue, comprising:
an endoscope, a catheter and a source of cryogen attached to said catheter.

78. The cryotherapy system of claim 77, wherein a proximal end of said catheter is connected to said cryogen source and a distal end of the catheter is placed in proximity to the tissue and cryogen flows from the source through the distal end to the tissue.

79. The cryotherapy system of claim 78, wherein the distal end of said catheter is guided to the tissue using a guiding device.

80. The cryotherapy system of claim 79, wherein the guiding device comprises a video camera and the distal end of the guiding device and/or the catheter is guided to the tissue by observing the distal end of the catheter and/or guiding device on a video monitor.

81. The cryotherapy system of claim 78, wherein the distal end of the catheter is adapted to spray the cryogen in a radial direction relative to the axis of the catheter.
